(19) United States
(12) Patent Application Publication
(10) Pub. No.: US 2026/0363981 A1
(43) Pub. Date: Nov. 19, 2026

(54) PERSONAL CARE COMPOSITIONS

(71) Applicant: ELEMENTIS SPECIALTIES, INC., East Windsor, NJ (US)

(72) Inventors: Kimberly BURCH, East Windsor, NJ (US); Melissa Marie FLECKENSTEIN, East Windsor, NJ (US); Erik GUNDERMAN, East Windsor, NJ (US)

(21) Appl. No.: 19/158,770

(22) PCT Filed: Feb. 23, 2024
(86) PCT No.: PCT/US2024/017060

§ 371 Date: Aug. 21, 2025

Related U.S. Application Data
(60) Provisional application No. 63/448,076, filed on Feb. 24, 2023.

(51) Int. Cl. A61K 8/37; A61K 8/31; A61Q 1/06; A61Q 1/10; A61Q 5/06; A61Q 15/00; A61Q 17/04; A61Q 19/10

(52) U.S. Cl. CPC A61K 8/375; A61K 8/31; A61Q 1/06; A61Q 1/10; A61Q 5/06; A61Q 15/00; A61Q 17/04; A61Q 19/10; A61K 2800/56; A61K 2800/651; A61K 2800/874

(57) ABSTRACT

A composition for personal care is described. The composition comprises: at least one personal care ingredient; and a rheological gel agent comprising: (i) an organoclay; (ii) a glyceryl ester activator; and (iii) a carrier. Also described is a method of making a composition and a method of applying a composition. A rheological gel agent for a composition for personal care is also described.

This application claims priority under 35 U.S.C. § 119 (e) from U.S. Provisional Patent Application No. 63/448,076 entitled “PERSONAL CARE COMPOSITIONS”, filed on Feb. 24, 2023, the contents of which are incorporated herein by reference in their entirety.

### FIELD OF THE INVENTION

The invention relates to compositions for personal care comprising rheological gel agents. The invention also relates to rheological gel agents for a composition for personal care. The invention has a particular, but not exclusive, application to the treatment and/or modification of the skin, lips, and/or keratin fibres. The invention also relates to a method for making a composition for personal care.

### BACKGROUND

Rheological gel agents are typically used to provide viscosity control, flow, levelling and other rheological properties to compositions.

Organoclays can also function as rheology modifiers in non-aqueous systems by building the viscosity of low to medium polarity carriers like mineral oil, fats, oils and other aliphatic compounds. Organoclays provide beneficial rheological modifications for the oil phase of emulsions, which leads to enhanced product stability, and increased viscosity without overloading the formula with excessively high levels of long chain fatty alcohols.

Organoclays are typically prepared by the reaction of an organic cation, in particular a quaternary ammonium ion, with a clay in various methods known in the art. If the organic cation contains at least one alkyl group containing at least 8 to 22 carbon atoms, then such organoclays have the property of increasing viscosity in organic based systems. The viscosity increasing properties can be modified by changing the substituents of the quaternary ammonium ion. For example, reports describe that the viscosity efficiency of organoclays was increased by substituting an alkyl group of the quaternary ammonium ion with a 2-hydroxyethyl group, a polyoxyethylene group and ester groups.

### SUMMARY OF THE INVENTION

According to a first aspect, the invention provides a composition for personal care. The composition comprises:
at least one personal care ingredient; and
a rheological gel agent comprising or consisting essentially of:
(i) an organoclay;
(ii) a glyceryl ester activator; and
(iii) a carrier.

It has been advantageously found that personal care compositions having improved rheological profiles are surprisingly obtained when formulated to include a specific type of rheological gel agent.

According to a second aspect, the invention provides a method of making a composition according the first aspect described herein. In the method, the rheological gel agent and the at least one personal care ingredient are incorporated into an oil phase and/or are subjected to shear mixing.

According to a third aspect, the invention provides a method of applying a composition according to the first aspect described herein. In the method, a shear stress is applied to the composition during application across the skin, lips, and/or keratin fibres.

According to a fourth aspect, the invention provides a rheological gel agent for use in a composition for personal care. The rheological gel agent comprises or consists essentially of:
(i) an organoclay;
(ii) a glyceryl ester activator; and
(iii) a carrier.

In the fourth aspect, the rheological gel agent is as defined for the rheological gel agent according to the first aspect described herein.

### DETAILED DESCRIPTION

The invention relates to the use of a rheological gel agent in a composition for personal care. It has been advantageously found that the rheological gel agent imparts improved rheological profiles to personal care composition, while being suitable for use in a personal care and being compatible with personal care ingredients.

Accordingly, the invention provides a composition for personal care. Thus, the composition is for use as a personal care product. For example, the composition is a home, cosmetic or beauty formulation and/or product.

The term “personal care” or “personal care product” as used herein refers to uses or products that are for typical consumer uses, including home, beauty, cleaning and cosmetic applications, formulations and/or products. For example, compositions for personal care include products such as Nail Colour Cosmetics, Deodorants, Lip Colour Cosmetics-Lip Colour, Face Colour Cosmetics-Foundations/Fluid Illuminators, Face/Neck Care, Multi-Use Face Colour Cosmetics-Concealer, Eye Colour Cosmetics-Eye Lash, Sun-Sun/Sunbed Exposure, Face Colour Cosmetics-Primer, Eye Colour Cosmetics-Eye Liner, Eye Colour Cosmetics-Eye Shadow, Eye Colour Cosmetics-Eye Brow, Lip Care, Face Colour Cosmetics-Blush, Lip Colour Cosmetics-Lip Liner, Hand/Nail Care, Shampoo, Sets, Eye Care, Body Care, Bar Soap, Foot Care, Hair Colorants, Face-Cleansers, Shower Products, Body Colour Cosmetics, Hair Styling, Face Colour Cosmetics-Bronzer, Face Colour Cosmetics-Powder, Anti-Fungal, Women's Fragrances, Feminine Hygiene Products, Sun-Self-Tanning, Hair Treatments, Skin Conditions, Men's Fragrances, Nail Enamel Removers, Shaving Preparations, Sun-After Sun, Dental Ancillaries, Eye-Cleansers, Pet Products, Antiseptic Treatments, Conditioner, Depilatory Products, Bath Additives, Shoe Care, Toothpaste, Decongestive, Cough, Cold & Flu Relief, Ear, Eye & Mouth Care, Plasters & Bandages, Other Healthcare, Aerosol, roll-on or stick based antiperspirant or deodorants, creams of oil in water and water in oil based emulsions for skin and sun care applications, lipstick and makeup cosmetics, haircare products such as shampoo, conditioner, styling or reactive hair care compositions.

In general, the term “personal care”, including when used in the context of the term “personal care product”, refer to a non-therapeutic uses, such as non-therapeutic, cosmetic uses.

The invention provides a composition for personal care. The composition comprises at least one personal care ingredient.

In certain embodiments, the personal care ingredient is selected from the group consisting of an antioxidant; an antiperspirant; a bulking agent; a cleansing agent; a colorant; a deodorizing agent; an emulsifier; an exfoliant; an eyelash conditioning agent; a film former; a foaming agent; a hair and/or skin conditioning agents; a humectant; a moisturizer; a perfuming agent; a skin protecting agent; a slip modifier; a smoothing agent; a surfactant; a tanning agent; a UV filter/absorber and a combination of two or more thereof. For the avoidance of doubt, the personal care ingredient is not water.

Additionally, or alternatively, the personal care ingredient is selected from the group consisting of fatty substances, organic solvents, oil structurants, surfactants, emulsifiers, thickeners, organic cationic deposition polymers, demulcents, opacifiers, additional colorants, effect pigments, additional stabilizers, emollients, antifoaming agents, moisturizing agents, antioxidants, vitamins, peptides, amino acids, botanical extracts, particulates, perfumes, preservatives, polymers, fillers, sequestrants, propellants, alkalinizing or acidifying agents, or mixtures thereof. In certain embodiments, one or more additional personal care ingredients customarily formulated into cosmetics or other compositions according to the invention also is/are present.

Typically, the personal care ingredient is a non-therapeutic ingredient, such as a non-therapeutic, cosmetic ingredient.

Clays, such as smectite clay, are commercially important minerals and are known in the art. Appropriately processed, smectite clays are excellent viscosifiers, binders, film formers, fabric softeners, and retention aid additives. These clays are 2:1 type layer silicates with an expandable structure. They are highly colloidal and may readily swell in water to form viscous, thixotropic gels, which renders the clays useful as viscosity builders in many industries. For instance, they are used to provide rheological control in compositions for industrial applications, such as coatings (e.g. paints).

Smectite clays (such as hectorite clay and bentonite clay) are products that are formed by, for instance, the decomposition of igneous rocks. Therefore, clays are relatively abundant in the environment, but their chemical composition may vary from deposit to deposit. These chemical variations result in for instance, differences in clay layer charge composition and density, impurity content and crystallite sizes. To counteract such variations, some smectite-like clays have been synthesized on an industrial scale. Such synthetic clays are uniform in charge density and are virtually impurity free. As a result, synthetic clays behave very differently from natural clays when used as, for instance, a rheological additive. A crude natural smectite clay includes both clay and impurities. In certain embodiments, such a crude clay is beneficiated or purified so that some or all of the impurities in the crude clay have been removed.

Smectite clays are layered, platy, hydrophilic silicate materials. In the dry state, several nano-sized clay layers are normally stacked on top of each other and these stacks, or tactoids, are agglomerated into particles. However, the platelets spontaneously separate from each other when dry clay powder is dispersed in water. This “delamination of layers” is at times also referred to as “exfoliation of layers.” Smectite clay layers carry a net negative charge on the platelets that is neutralized by metal cations that are positioned on the surfaces of the platelets.

An organoclay is formed when the metal cations are exchanged with organic cations. This reaction is partially completed or driven to completion. Organic surface treatment is often necessary to improve the compatibility of the clay with organic systems. Similar to “pristine” inorganic clays in water, organoclays can delaminate in organic systems (solvents, polymers): i.e. the clay layers that are now decorated with organic cations are separated from each other when they are exfoliated in said systems.

In general, the organoclay is a phyllosilicate clay in which interlayer metal cations are exchanged for organic cations. Thus, the organoclay is a phyllosilicate clay having interlayer organic cations. The organic cations are typically quaternary ammonium ions.

Typically, the organoclay is a smectite clay (e.g. the phyllosilicate clay is a smectite clay). In certain embodiments, the smectite clay is a montmorillonite, a bentonite, a hectorite, a saponite, a stevensite and/or a beidellite clay.

It is preferred that the smectite clay is a hectorite clay, a bentonite clay or a mixture thereof. In certain embodiments, the smectite clay is a hectorite clay. In certain embodiments, the smectite clay is a bentonite clay. More preferably, the smectite clay is a hectorite clay.

The quaternary ammonium ions typically have a formula of [N—R¹R²R³R⁴]+, wherein each of R¹, R² and R³ is independently a C₁-C₂₂ alkyl group, such as a C₁-C₁₈ alkyl group, particularly a C₁-C₁₂ alkyl group, preferably a C₁-C₈ alkyl group.

R⁴ is selected from a linear C₁-C₆ alkyl group, a C₆-C₁₀ aryl group and a combination of two or more thereof.

It is preferred that R⁴ is selected from a linear C₁-C₆ alkyl group, a C₆-C₁₀ aryl group and a C₆-C₁₀ aryl-C₁-C₆ alkyl group in which the C₁-C₆ alkyl moiety is linear (e.g. a C₆-C₁₀ aryl-linear-C₁-C₆ alkyl group). More preferably, R⁴ is a phenyl group, a benzyl group, a methyl group, an ethyl group, a propyl group, a butyl group, a pentyl group or a hexyl group. Even more preferably, R⁴ is a benzyl group, a methyl group, an ethyl group, a propyl group, a butyl group, a pentyl group or a hexyl group.

Typically, one or more of R¹, R² and R³ is a mixture of branched C₁₂-C₂₂ alkyl groups. Each branched alkyl group has a linear backbone and one or more branching C₁-C₃ alkyl groups, wherein the branching C₁-C₃ alkyl groups are distributed at different carbon positions along the linear backbone of the branched alkyl group.

In one example, each branched alkyl group has a total of 12 to 18 carbon atoms, preferably 14 to 18 total carbon atoms.

It is preferred that at least one of R² and R³ is not a branched alkyl group. Typically, at least one of R² and R³ or both R² and R³ is a linear C₁-C₂₂ alkyl group, preferably a linear C₁-C₁₂ alkyl group, more preferably a linear C₁-C₆ alkyl group or a linear C₇-C₁₁ alkyl group, particularly a linear C₁-C₆ alkyl group.

In one example, one or more of R² and R³ is methyl and R⁴ is benzyl. In another example, R², R³ and R⁴ is each methyl.

When R¹ is a branched alkyl group, the branched alkyl group has an average number of branches, per branched alkyl group, of at least 0.7, such as an average number of branches per branched alkyl group of from 0.7 to 7, preferably from 0.7 to 5, more preferably from 0.7 to 3. A methyl branch is typically at least 50% of the branching alkyl groups based on the total number of branches.

Additionally, or alternatively, when R¹ is a branched alkyl group, the branched alkyl group has a distribution of branching points distributed along the linear backbone of the branched alkyl group ranging from a 2 carbon atom position on the linear backbone, counting from a 1 carbon atom position which is bonded to N⁺, to a ω-2 carbon atom position, where w is a terminal carbon atom position on the linear backbone. In such examples, a methyl branch is at least 50% of the branching alkyl groups based on the total number of branches.

In one example, the linear backbone, of the R¹ branched alkyl group, contains less 0.5 atom % of quaternary carbon atoms. In another example, the linear backbone, of the R¹ branched alkyl group, is substantially free of quaternary carbon atoms.

Typically, the organoclay comprises sufficient quaternary ammonium ions to satisfy 50 to 150 percent of phyllosilicate cation exchange capacity (e.g. 50 to 100 percent), such as 90 to 140 percent of phyllosilicate cation exchange capacity (e.g. 90 to 100 percent), particularly 95 to 130 percent of phyllosilicate cation exchange capacity. When the quaternary ammonium ions are in excess of the exchange capacity, then the positive charge of the quaternary ion is balanced by an inorganic anion or organic anion or anionic polymer. Examples of organic anions are found in U.S. Pat. No. 5,718,841 which is incorporated by reference in its entirety herein.

In certain embodiments, the organoclay is present in a total amount from about 5 wt. % to about 30 wt. %, with respect to the total weight of the rheological gel agent, preferably from about 5 wt. % to about 25 wt. %, and more preferably from about 8 wt. % to about 20 wt. %.

As used herein, the term “glyceryl ester activator” refers to a compound that is an ester of glycerol. In other words, at least one of the alcohol (e.g. hydroxy) groups in glycerol is esterified (e.g. forms an ester group). Thus, the glyceryl ester activator includes at least one glyceryl group and at least one ester group.

Preferably, the or each ester group of the glyceryl ester activator is a fatty acid ester group. In other words, the ester group is derived from a fatty acid (e.g. by reaction with an alcohol group of the glycerol).

The term “aliphatic”, as used herein, means a substituted or unsubstituted straight-chain, branched or cyclic hydrocarbon. The hydrocarbon is either completely saturated or is unsaturated (preferably only contains one or more double bonds) and is not aromatic.

Typically, short-chain fatty acids (SCFA) are fatty acids with aliphatic chains having 5 or fewer carbons (e.g. butyric acid), medium-chain fatty acids (MCFA) are fatty acids with aliphatic chains having 6 to 12 carbons, long-chain fatty acids (LCFA) are fatty acids with aliphatic chains having 13 to 21 carbons and very long chain fatty acids (VLCFA) are fatty acids with aliphatic chains having 22 or more carbons.

Saturated fatty acids have no C═C double bonds. Unsaturated fatty acids have one or more C═C double bonds, each of which have a cis or a trans arrangement.

As defined herein, the amount of carbon atoms present in the aliphatic chain is the number of carbons extending from and including the carbonyl carbon of the fatty acid ester group. For example, glycerol stearate has an aliphatic chain having 18 carbon atoms.

It may be preferable that the glyceryl ester activator comprises one or more fatty acid ester groups, wherein each fatty acid ester group has an aliphatic chain having at least 8 carbon atoms. Preferably the aliphatic chain has from 8 to 22 carbon atoms, more preferably from 8 to 18 carbon atoms.

In certain embodiments, the glyceryl ester activator comprises one fatty acid ester group, wherein each fatty acid ester group has an aliphatic chain having from 8 to 22 carbon atoms, more preferably from 8 to 18 carbon atoms.

Preferably, the glyceryl ester activator comprises one or more fatty acid esters (more preferably one fatty acid ester), wherein the fatty acid ester group has an aliphatic chain having from 8 to 18 carbon atoms and is linear, saturated and unsubstituted.

In certain embodiments, the glyceryl ester activator is present in a total amount from about 0.5 wt. % to about 8.0 wt. %, with respect to the total weight of the rheological gel agent, preferably from about 0.5 wt. % to about 5 wt. %, more preferably from about 0.5 wt. % to about 3 wt. %.

In certain embodiments, the glyceryl ester activator is selected from the group consisting of glyceryl arachidate, glyceryl behenate, glyceryl caprate, glyceryl caprylate, glyceryl cocoate, glyceryl erucate, glyceryl hydroxystearate, glyceryl isostearate, glyceryl laurate, glyceryl linoleate, glyceryl myristate, glyceryl oleate, glyceryl palmitate, glyceryl ricinoleate, glyceryl stearate, and a mixture of two or more thereof. Typically, the glyceryl ester activator is selected from the group consisting of glyceryl arachidate, glyceryl behenate, glyceryl caprate, glyceryl caprylate, glyceryl erucate, glyceryl hydroxystearate, glyceryl isostearate, glyceryl laurate, glyceryl linoleate, glyceryl myristate, glyceryl oleate, glyceryl palmitate, glyceryl ricinoleate, glyceryl stearate, and a mixture of two or more thereof. Preferably, the glyceryl ester activator comprises glycerol stearate. More preferably, the glyceryl ester activator is glycerol stearate.

The specific glycerol ester activator that is used in the rheological gel agent depends on the carrier.

In some examples, the glyceryl ester activator has a weight average molecular weight (MW) of about 215 to 415, preferably of about 250 to 375.

The glyceryl ester is included in the rheological gel agent to activate the organoclay. In general, the amount by weight of glyceryl ester needed to activate the organoclay is less than or equal to half of the amount by weight of the organoclay.

The rheological gel agent typically comprises a total amount of the organoclay to the total amount of the glyceryl ester activator in a weight ratio of from 40.0:1 to 2.0:1, preferably from 35.0:1 to 3.0:1, such as from 30.0:1 to 3.5:1, more preferably 25.0:1 to 4.0:1.

The composition for personal care typically comprises a total amount of the organoclay to the total amount of the glyceryl ester activator in a weight ratio of from 40.0:1 to 2.0:1, preferably from 35.0:1 to 3.0:1, such as from 30.0:1 to 3.5:1, more preferably 25.0:1 to 4.0:1. In certain embodiments, all of the glyceryl ester ingredient in the composition is provided as an activator in the rheological gel agent.

The rheological gel agent and/or the composition for personal use preferably do not include an activator that is not a glyceryl ester. For example, the rheological gel agent and/or the composition for personal use do not include propylene carbonate or ethanol.

The carrier (e.g. a solvent or surfactant) is a single-phase system or a multiphase system. In certain embodiments, multiphase systems include an oil in water emulsion or a water in oil emulsion. It is preferred that the carrier is a single-phase system.

The carrier is preferably non-aqueous.

In certain embodiments, the carrier is selected from the group consisting of triglyceride oil; an ester; a linear and/or a branched fatty alcohol; a linear and/or branched hydrocarbon; a silicone and a mixture of two or more thereof. The ester is preferably not a glyceryl ester.

In certain embodiments, the carrier is present in an amount from about 60 wt. % to about 95 wt. %, with respect to the total weight of the rheological gel agent, preferably about 60 wt. % to about 90 wt. %, In some examples, the 62 wt. % to about 87 wt. %.

It is preferred that the carrier and/or the rheological gel agent is not an emulsion.

In some examples, the rheological gel agent comprises or consists essentially of: about 5 wt. % to about 30 wt. % of the organoclay; about 0.5 wt. % to about 8.0 wt. % of the glyceryl ester activator; and about 60 wt. % to about 95 wt. % of the carrier.

In certain embodiments, the rheological gel agent is present in an amount from about 0.5 wt. % to about 20 wt. % with respect to the total weight of the composition, preferably about 1 wt. % to 15 wt. %

In certain embodiments, the rheological gel agent has an initial viscosity between about 200,000 cP to about 20,000,000 cP, preferably about 250,000 to about 20,000,000, more preferably about 500,000 to about 15,000,000. Initial viscosity is measured using a Brookfield viscometer using a spindle and rotation setting to calculate the viscosity, as conventionally measured for compositions containing an organoclay.

The rheological gel agent typically has a pH of from 6.0 to 8.0, preferably 6.5 to 7.5 (e.g. about 7.0).

In certain embodiments, the organoclay is dispersed within the carrier (preferably an oil phase carrier) and activated with an optimum level of a glyceryl ester activator. It has been advantageously found that the glyceryl ester activator improves the dispersion of the organoclay within the carrier. In certain embodiments, the organoclay is dispersed using high-shear equipment. In certain embodiments, the organoclay is at least partially dispersed within the carrier.

Typically, the organoclay is dispersed within the carrier at a concentration of about 5% to about 35%, preferably about 5 to about 25%, more preferably about 8% to about 20%.

The gels produced with the glyceryl activator have the benefit of being more natural and have lower irritation potential, particularly when compared to standard activators used with organoclays that are not natural and are irritant to humans. The gels are softer and easier to disperse, and provide a better skin feel. The glyceryl ester also has formulation benefits, such as providing good stability, as well as providing benefits in use to skin and hair.

In certain embodiments, the composition according to the first aspect of the invention described herein is used in a method of treatment and/or modification of the skin, lips, and/or keratin fibres. In certain embodiments, the treatment and/or modification is moisturization, UV protection, and/or pigment deposition.

In certain embodiments, the method of treatment is a therapeutic method or a non-therapeutic method. It is preferred that the method is a non-therapeutic method, such as a non-therapeutic, cosmetic method.

According to a second aspect, the invention provides a method of making a composition according the first aspect described herein. In the method, the rheological gel agent and the at least one personal care ingredient are incorporated into an oil phase and/or are subjected to shear mixing.

According to a third aspect, the invention provides a method of applying a composition according the first aspect described herein. In the method, a shear stress is applied to the composition during application across the skin, lips, and/or keratin fibres.

The compositions of the present invention have a beneficial use for personal care compositions suitable to exhibit a variety of hereto unknown properties and satisfy various aesthetic and application criteria. For example, the compositions of this invention provide for rheology control, prevent sag of ingredients and impart a desirable feel characteristic to consumer formulations in a variety of applications areas.

Compositions for personal care can be divided into two product categories: leave-on and rinse-off. Many of them are indeed fragranced to bring pleasant aromas as an important consumer benefit. While leave-on products such as creams and lotions definitely have the challenge just described, rinse-off products such as shampoo, conditioner, and body wash face even more challenge(s) due to the loss of the main fragrance content during washing/cleansing processes. Therefore, as a key unmet consumer need, fragrance retention or prolongation has been intensively researched in personal care and cosmetic industries. In certain embodiments, the compositions are leave-on compositions or wash-off compositions.

In certain embodiments, the composition is a home, cosmetic or beauty formulation and/or product. The glyceryl esters are natural and non-irritating nature, in comparison to the activators that are used in combination with organoclays for industrial applications.

Cosmetic products range from pressed dry powders to suspensions in liquids; from single-phase solid or liquid formulations to emulsions. The levels of use of the composition depends on the system and the desired performance criteria. The properties imparted by the organoclay dispersed compositions benefit different cosmetics in different ways. The advantages of using the organoclay compositions of the current invention are for instance for: Stick products (lipstick and antiperspirant): to maintain homogeneity in molten and setting stages; gain improved pay-out; increase high-temperature integrity; eliminate oil migration; reduce “creasing” around lips and eyes.

Mascara: to improve film-build; increase water-resistance; eliminate oil migration.

Emulsions: to elevate drop-point temperature of water-in-oil creams; eliminate syneresis; improve emulsion stability; enable cold-process emulsification; create novel viscosity effects.

UV Sunscreen: to eliminate ultrafme TiO2 skin-whitening problems; enhance sun protection factor (SPF): optimise use of sunscreen actives; reduce separation and settlement.

Antiperspirant Aerosols: to provide uniform distribution of active ingredients; soft and dry skin feel; significant less whitening; reduced separation and settlement.

For the avoidance of doubt, the expression “consists essentially of” as used herein limits the scope of a feature to include the specified materials, and any other materials or steps that do not materially affect the basic and novel characteristics of that feature, such as, for example, minor impurities. The expression “consists essentially of” embraces the expression “consisting of”.

The entire disclosure of each document cited herein is hereby incorporated herein by reference. All references cited in this disclosure are incorporated by reference to the same extent as if each reference had been incorporated by reference in its entirety individually.

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

A number of embodiments of the disclosure have been described. Nevertheless, it will be understood that various modifications may be made without departing from the spirit and scope of the present disclosure. Accordingly, other embodiments are within the scope of the following claims.

### EXAMPLES

The invention is illustrated by the following non-limiting examples. All parts and percentages are given by weight unless otherwise stated. The examples disclosed herein demonstrate various compositions for personal care.

It will be understood that all tests and physical properties listed have been determined at atmospheric pressure and ambient temperature (i.e. about 23 °C), unless otherwise stated, or unless otherwise stated in the referenced test method and/or procedure.

### Example 1: Preparation of Rheological Gel Agents

The rheological gel agent was prepared by blending at high shear the following components:
(i) Organoclay
(ii) Glyceryl ester activator; and
(iii) Carrier (e.g. solvent).

The completed blends were than tested for initial viscosity for effectiveness and applicability. The results can be seen in table 1.

TABLE 1
Glyceryl ester rheological gel agents.

| Activator | Activator Level % | Solvent | Bentone 38 V | Initial Viscosity, cP |
|---|---|---|---|---|
| Glyceryl Oleate (90%) | 1% | Hemisqualane | 24% | 2906400 |
| Glyceryl Oleate (90%) | 1% | Hemisqualane | 24% | 14068000 |
| Glyceryl Oleate (90%) | 1% | Hemisqualane | 24% | 897600 |
| Glyceryl Stearate | 1% | Hemisqualane | 18% | 2814000 |
| Glyceryl Oleate (90%) | 1% | Coco-caprylate/Caprate | 18% | 2162000 |
| Glyceryl Tribehenate | 1% | Hemisqualane | 18% | 2175200 |
| Glyceryl Laurate | 1% | Hemisqualane | 18% | 14160000 |
| Glyceryl Caprylate | 1% | Hemisqualane | 18% | 11910000 |
| Glyceryl Triisocaprylate | 1% | Hemisqualane | 18% | 1173000 |
| Glyceryl Tricaprylin | 1% | Hemisqualane | 18% | 1782000 |
| Sorbitan Oleate | 1% | Hemisqualane | 18% | 3190000 |
| Glyceryl Oleate (90%) | 1% | Hemisqualane | 9% | 997600 |
| Glyceryl Oleate (90%) | 1% | Coco-caprylate/Caprate | 10% | 552000 |
| Glyceryl Oleate (90%) | 1% | Coco-caprylate/Caprate | 10% | 584800 |

### Example 2: Optimization Example of Glyceryl Oleate

The rheological gel agent can be made from many glyceryl ester activators (e.g. mono glyceryl ester activators) and each system can be optimized. The following examples were prepared by the same procedure as Example 1 and were tested for initial viscosity to optimize the composition of the blend.

TABLE 2
Glyceryl oleate rheological gel agents.

| Activator | Activator Level % | Solvent | Bentone 38 V | Initial Viscosity, cP |
|---|---|---|---|---|
| Glyceryl Oleate (90%) | 2.25% | Hemisqualane | 24% | 12890000 |
| Glyceryl Oleate (90%) | 1.75% | Hemisqualane | 24% | 13992000 |
| Glyceryl Oleate (90%) | 1.25% | Hemisqualane | 24% | 3280000 |
| Glyceryl Oleate (90%) | 0.75% | Hemisqualane | 24% | 720800 |
| Glyceryl Oleate (90%) | 1.00% | Hemisqualane | 24% | 2906400 |
| Glyceryl Oleate (90%) | 1.25% | Hemisqualane | 24% | 16004000 |
| Glyceryl Oleate (90%) | 0.87% | Hemisqualane | 24% | 1870000 |
| Glyceryl Oleate (90%) | 1.00% | Hemisqualane | 24% | 14068000 |
| Glyceryl Oleate (90%) | 1.00% | Hemisqualane | 24% | 897600 |
| Glyceryl Oleate (90%) | 0.87% | Hemisqualane | 24% | 13810000 |
| Glyceryl Oleate (90%) | 1.00% | Coco-caprylate/Caprate | 18% | 2162000 |
| Glyceryl Oleate (90%) | 2.25% | Hemisqualane | 9% | 68000 |
| Glyceryl Oleate (90%) | 1.00% | Hemisqualane | 9% | 997600 |
| Glyceryl Oleate (90%) | 2.25% | Hemisqualane | 9% | 337600 |
| Glyceryl Oleate (90%) | 3.00% | Coco-caprylate/Caprate | 10% | 392500 |
| Glyceryl Oleate (90%) | 1.00% | Coco-caprylate/Caprate | 10% | 552000 |
| Glyceryl Oleate (90%) | 1.00% | Coco-caprylate/Caprate | 10% | 584800 |

### Example 3: Oil in Water Inorganic Sunscreen (Application Example)

TABLE 3
Ingredients.

| | |
|---|---|
| Ingredient | % w/w |

Phase A

| | |
|---|---|
| Rheological gel agent (Glyceryl | 3 |
| Oleate, Hemisqualane, | |
| Disteardimonium Hectorite) | |
| C12-15 Alkyl Benzoate | 10 |
| Cetearyl Ethylhexanoate | 5 |
| Cetyl Alcohol | 3 |
| Sorbitan Stearate | 2.2 |
| Beeswax | 0.5 |

Phase B

| | |
|---|---|
| Zinc Oxide | 7 |
| Titanium Dioxide | 1.2 |

Phase C

| | |
|---|---|
| De-ionised Water | qs |
| Vegetable Glycerin | 8 |
| Polysorbate 60 | 3.3 |

Phase D

| | |
|---|---|
| Phenoxyethanol | 0.2 |

Procedure: Heat Phase A together to 75 °C. Transfer to a Silverson homogeniser, and with mixing gradually add Phase B. Mix until fully dispersed. In a separate container warm Phase C to 75 °C. Using high shear mixing (e.g. Silverson homogeniser), slowly add Phase A+B to Phase C, continue to mix for several minutes. Transfer to propeller stirrer and commence cooling, until under 35 °C, add Phase D. Mix until uniform.

### Example 4: Water in Oil Cream (Application Example)

TABLE 4
Ingredients.

| | |
|---|---|
| Ingredient | % w/w |

Phase A

| | |
|---|---|
| Rheological gel agent (Glyceryl | 9 |
| Laurate, Caprylic/Capric Triglyceride, | |
| Stearalkonium Hectorite) | |
| Mineral Oil | 16 |
| Polyglyceryl-3 Diisostearate | 3 |
| Paraffin Wax | 3 |

Phase B

| | |
|---|---|
| Deionised Water | qs |

Procedure: Heat the Phase A and Phase separately to 65-70 °C. Add Phase B to Phase A with stirring. Stir to cool.

### Example 5: Mascara (Application Example)

TABLE 5
Ingredients.

| | |
|---|---|
| Ingredient | % w/w |

Phase A

| | |
|---|---|
| Rheological gel agent (Glyceryl Oleate, Coco- | 15 |
| caprylate/Caprate, Stearalkonium Hectorite) | |
| Cyclomethicone | 21 |
| C.I. 77499 and Dimethicone | 20 |
| Carnauba Wax | 1 |
| Glyceryl Stearate | 1 |

Phase B

| | |
|---|---|
| Deionised Water | qs |
| Glycerine | 4 |
| Propylene Glycol | 2 |
| Sorbitan Sesquioleate | 2 |
| VP/VA Copolymer | 1 |
| Panthenol | 1 |
| Tetrasodium EDTA | 0.1 |

Procedure: Mix Part A together and heat to 70-75 °C. Premix Part B and heat to 70-75 °C. Add Part A to Part B with high shear mixing (i.e. Silverson Homogeniser). Cool with propeller stirring.

### Example 6: Lip Gloss (Application Example)

TABLE 6
Ingredients.

| | |
|---|---|
| Ingredient | % w/w |

Phase A

| | |
|---|---|
| Rheological gel agent (Glyceryl Oleate, | 20 |
| Hemisqualane, Stearalkonium Hectorite) | |
| Polybutene | 64.1 |
| Octyldodecanol | 7 |
| Cera Carnauba | 4 |
| Tocopheryl Acetate | 0.5 |

Phase B

| | |
|---|---|
| Trihydroxystearin | 1 |

Phase C

| | |
|---|---|
| Pigments | 3.3 |

Procedure: Heat Phase A together to 85 °C, with mixing. Add Phase B and mix. Add Phase C and mix. Cool to 35 °C and with propeller stirring.

### Example 7: Lipstick (Application Example)

TABLE 7
Ingredients.

| | |
|---|---|
| Ingredient | % w/w |

Phase A

| | |
|---|---|
| Rheological gel agent (Glyceryl | 10 |
| Hydroxystearate, Ricinus communis seed | |
| oil, Stearalkonium Hectorite) | |
| Propylene Glycol Dicaprylate/Caprate | 31.4 |
| Castor Oil (Ricinus Communis) | 24 |
| Candelilla Cera and Paraffin | 8 |
| Ozokerite | 4 |

Phase B

| | |
|---|---|
| Trihydroxystearin | 1 |

Phase C

| | |
|---|---|
| Pigment (Ricinus Communis and Red 7 | 20 |
| Lake) | |

Procedure: Heat Phase A together to 85 °C. Bring temperature down to 80 °C. Add and mix in Phase B, using a homomixer at medium speed. Mix for 10 minutes at 80 °C. Add Phase C and mix for 5 minutes. Continue mixing at medium speed and lower temperature to 70 °C and add Phase D and mix for 5 minutes. Pour into molds.

### Example 8: Antiperspirant Roll-on

TABLE 8
Ingredients.

| | |
|---|---|
| Ingredient | % w/w |

Phase A

| | |
|---|---|
| Rheological gel agent (Glyceryl Myristate, | 50 |
| Isopropyl Myristate, Stearalkonium | |
| Hectorite) | |
| Cyclomethicone | 27 |
| Alcohol Denatured | 3 |

Phase B

| | |
|---|---|
| Cyclomethicone | 27 |
| Dimethicone | 3 |

Phase C

| | |
|---|---|
| Aluminium Chlorohydrate | 25 |

Procedure: Thoroughly disperse Phase A using a silverson homogeniser. Add Phase B to Phase A and continue mixing. With slow speed propeller stirring add Phase C to Phase A+B. Add Phase D to Phase A+B+C and mix until uniform.

### Example 9: Aerosol Antiperspirant

TABLE 9
Ingredients.

| | |
|---|---|
| Ingredient | % w/w |

Phase A

| | |
|---|---|
| Rheological gel agent (Glyceryl Stearate, | 2 |
| Cyclopentasiloxane, Disteardimonium | |
| Hectorite | |
| Cyclomethicone | 10 |
| Dimethicone | 2 |
| Alcohol Denatured | 0.5 |

Phase B

| | |
|---|---|
| Aluminium Chlorohydrate | 10 |

Phase C

| | |
|---|---|
| Propellant | 75.5 |

Procedure: Premix Phase A. Add Phase B to Phase A. Fill and charge with Phase C.

### Example 10: Hair Pomade

TABLE 10
Ingredients.

| | |
|---|---|
| Ingredient | % w/w |

Phase A

| | |
|---|---|
| Rheological gel agent (Glyceryl Isostearate, | 6 |
| Pentaerythrityl Tetraisostearate, | |
| Disteardimonium Hectorite) | |
| Cetearyl Alcohol | 11 |
| Mineral Oil | 7 |
| Cetearyl Ethylhexanoate | 6 |
| Beeswax | 5 |
| Glyceryl Stearate | 4 |
| Sorbitan Isostearate | 4 |

Phase B

| | |
|---|---|
| Cyclomethicone | 57 |

Procedure: Combine Part A and heat to 110 °C with mixing. Cool with mixing to 70 °C. Add Part B to main batch, with mixing. Cool with mixing.

### Example 11: Shower Gel

TABLE 11
Ingredients.

| | |
|---|---|
| Ingredient | % w/w |

Phase A

| | |
|---|---|
| Rheological gel agent (Glyceryl Cocoate, Cocos | 1 |
| Nucifera Oil, Disteardimonium Hectorite) | |
| Sodium Lauryl Sulphate | 30 |
| Coco Glucoside | 6 |
| PEG-60 Corn Glycerides | 5 |

Phase B

| | |
|---|---|
| Deionised Water | 50.5 |
| Panthenol | 0.5 |

Phase C

| | |
|---|---|
| Hydroxyethyl Cellulose | 2 |

Phase D

| | |
|---|---|
| Cocamidopropylbetaine | 3 |
| Glycol Stearate | 2 |

Procedure: Warm the Phase A together. Heat Phase B to 55 °C, then add Phase C with silverson homogenising. Add Phase A to Phase B slowly with stirring. Add Phase D with stirring. The temperature should be at 35 °C or below by this stage, adjust pH.

1. A composition for personal care, comprising:
at least one personal care ingredient; and
a rheological gel agent comprising:
(i) an organoclay;
(ii) a glyceryl ester activator; and
(iii) a carrier.

2. The composition according to claim 1, wherein the organoclay is a smectite clay.

3. The composition according to claim 2, wherein the smectite clay is a hectorite clay, a bentonite clay or a mixture thereof.

4. The composition according to claim 2, wherein the smectite clay is a hectorite clay.

5. The composition according to claim 1, wherein the rheological gel agent comprises the organoclay in a total amount of about 5 wt. % to about 30 wt. %.

6. The composition according to claim 1, wherein the organoclay is dispersed within said carrier at a concentration of about 5% to about 35%.

7. The composition according to claim 1, wherein the rheological gel agent comprises the glyceryl ester activator in a total amount of about 0.5 wt. % to about 8.0 wt. % of the glyceryl ester activator.

8. The composition according to claim 1, wherein the rheological gel agent comprises a total amount of the organoclay to the total amount of the glyceryl ester activator in a weight ratio of from 40.0:1 to 2.0:1, preferably from 35.0:1 to 3.0:1, such as from 30.0:1 to 3.5:1, more preferably 25.0:1 to 4.0:1.

9. The composition according to claim 1, wherein the glyceryl ester activator comprises at least one fatty acid ester group, wherein each fatty acid ester group has an aliphatic chain having from 8 to 22 carbon atoms.

10. The composition according to claim 1, wherein the glyceryl ester activator is selected from the group consisting of glyceryl arachidate, glyceryl behenate, glyceryl caprate, glyceryl caprylate, glyceryl cocoate, glyceryl erucate, glyceryl hydroxystearate, glyceryl isostearate, glyceryl laurate, glyceryl linoleate, glyceryl myristate, glyceryl oleate, glyceryl palmitate, glyceryl ricinoleate, glyceryl stearate, and a mixture of two or more thereof.

11. The composition according to claim 1, wherein the glyceryl ester activator is glycerol stearate.

12. The composition according to claim 1, wherein the glyceryl ester activator has a weight average molecular weight (MW) of about 215 to about 415.

13. The composition according to claim 1, wherein the rheological gel agent comprises the carrier in a total amount of about 60 wt. % to about 95 wt. %.

14. The composition according to claim 1, wherein the carrier is selected from the group consisting of a triglyceride oil; an ester; a linear and/or a branched fatty alcohol; a linear and/or branched hydrocarbon; a silicone and a mixture of two or more thereof.

15. The composition according to claim 1, wherein the rheological gel agent comprises or consists essentially of:
(i) about 5 wt. % to about 30 wt. % of the organoclay;
(ii) about 0.5 wt. % to about 8.0 wt. % of the glyceryl ester activator; and
(iii) about 62 wt. % to about 95 wt. % of the carrier.

16. The composition according to claim 1, wherein the rheological gel agent has an initial viscosity between about 200,000 cP to about 20,000,000 cP.

17. The composition according to claim 1, wherein the rheological gel agent is present in a total amount from about 0.5 wt. % to 20 wt. %.

18. The composition according to claim 1, wherein the personal care ingredient is selected from the group consisting of an antioxidant; an antiperspirant; a bulking agent; a cleansing agent; a colorant; a deodorizing agent; an emulsifier; an exfoliant; an eyelash conditioning agent; a film former; a foaming agent; a hair and/or skin conditioning agents; a humectant; a moisturizer; a perfuming agent; a skin protecting agent; a slip modifier; a smoothing agent; a surfactant; a tanning agent; and a UV filter/absorber.

19. The composition according to claim 1, for use in the treatment and/or modification of the skin, lips, and/or keratin fibres.

20. The composition according to claim 19, wherein the treatment and/or modification is moisturization, UV protection, and/or pigment deposition.

21. The composition according to claim 1, wherein the composition is a home, cosmetic or beauty formulation and/or product.

22. Use of a composition according to claim 1 in a method of treatment and/or modification of the skin, lips, and/or keratin fibres.

23. Use of a composition according to claim 22, wherein the treatment and/or modification is moisturization, UV protection, and/or pigment deposition.

24. A method of making a composition according to claim 1, wherein the rheological gel agent and the at least one personal care ingredient are incorporated into an oil phase and/or are subjected to shear mixing.

25. A method of applying a composition according to claim 1, wherein a shear stress is applied to the composition during application across the skin, lips, and/or keratin fibres.

26. A rheological gel agent for a composition for personal care, comprising:
(i) an organoclay;
(ii) a glyceryl ester activator; and
(iii) a carrier;
preferably wherein the rheological gel agent is as defined in claim 15.
